(19) United States
(12) Patent Application Publication
(10) Pub. No.: US 2014/0127315 A1
(43) Pub. Date: May 8, 2014

(54) Caudal Salve

(71) Applicant: Sarah L. Brown, Windsor, ME (US)

(72) Inventor: Sarah L. Brown, Windsor, ME (US)

(21) Appl. No.: 14/073,864

(22) Filed: Nov. 6, 2013

Related U.S. Application Data
(60) Provisional application No. 61/723,035, filed on Nov. 6, 2012.

(51) Int. Cl. A61K 36/534; A61K 36/28; A61K 31/192; A61K 31/07; A61K 31/355; A61K 31/20; A61K 35/64; A61K 31/05

(52) U.S. Cl. CPC A61K 36/534; A61K 35/644; A61K 36/28; A61K 31/05; A61K 31/07; A61K 31/355; A61K 31/20; A61K 31/192
USPC 424/539

(57) ABSTRACT

A caudal salve is a topical treatment for irritated skin around a user's posterior. The caudal salve includes a plurality of ingredients that each has their own unique beneficial properties. The plurality of ingredients includes a quantity of bees wax, a quantity of shea butter, a quantity of coconut oil, a quantity of yarrow-plant oil, a quantity of calendula-flower oil, a quantity of menthol, and a quantity of peppermint oil. The bees wax, the shea butter, and the coconut oil are typically used as the base materials for the caudal salve. The yarrow-plant oil, the calendula-flower oil, the menthol, and the peppermint oil are typically used as the supplemental yet active materials for the caudal salve.

The current application claims a priority to the U.S. Provisional Patent application Ser. No. 61/723,035 filed on Nov. 6, 2012.

### FIELD OF THE INVENTION

The present invention relates generally topical creams. More specifically, the present invention is an all natural cream that the user can apply to their bottom for itching, burning, and pain relief.

### BACKGROUND OF THE INVENTION

In the field of proctology, there are many patients that desire relief for their bottom from uncomfortable symptoms such as pain, itching, and burning. Many individual ingredients or solutions have been used in the past but are limited in their effectiveness. It is therefore an object of the present invention to introduce an all natural cream that is a combination of effective and soothing ingredients to help alleviate the uncomfortable symptoms that is experienced by patients.

### BRIEF DESCRIPTION OF THE DRAWINGS

FIG. 1 is a block diagram depicting the ingredients of the present invention.

FIG. 2 is a block diagram depicting the ratio of the ingredients within the present invention.

FIG. 3 is a block diagram depicting the topically beneficial constituents of shea butter.

FIG. 4 is a block diagram depicting the topically beneficial constituents of coconut oil.

### DETAILED DESCRIPTIONS OF THE INVENTION

All illustrations of the drawings are for the purpose of describing selected versions of the present invention and are not intended to limit the scope of the present invention.

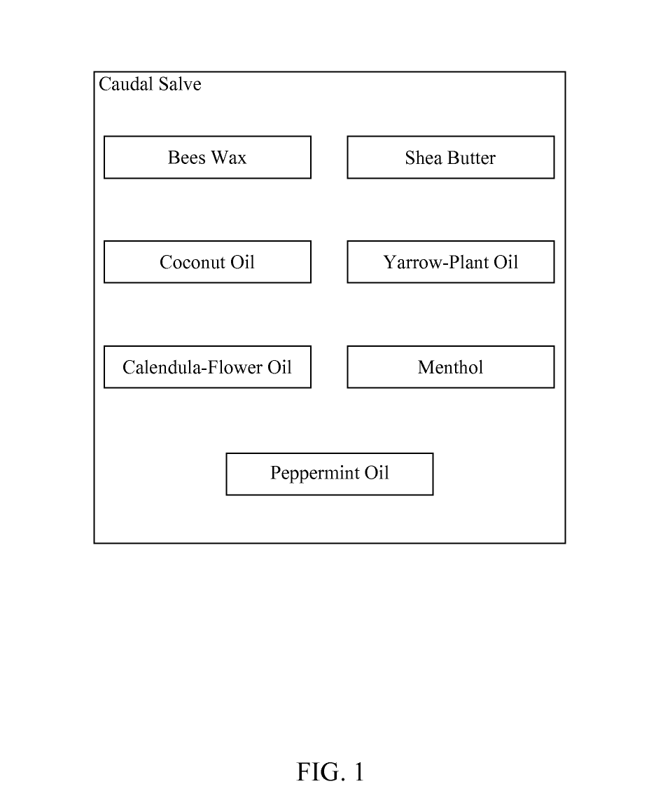

The present invention is a caudal salve that is used as a topical treatment to soothe and relieve irritation near a user's posterior. The present invention is constituted to also heal the irritation near a user's posterior. As can be seen in FIG. 1, the present invention mainly comprises a quantity of bees wax, a quantity of shea butter, a quantity of coconut oil, a quantity of yarrow-plant oil, a quantity of calendula-flower oil, a quantity of menthol, and a quantity of peppermint oil. Bees wax is a natural wax that is produced by honey bees and can be used to help moisturize the skin. Shea butter is a natural fat that is extracted from the nuts of a shea tree and can be used to help moisturize the skin and be used as an anti-inflammatory on the skin. Coconut oil is a natural oil that is extracted from the coconut kernels and can also be used to help moisturize the skin. Yarrow-plant oil is a natural oil extracted from a common yarrow, which is a flowering plant, and can be used for medicinal purposes because of its astringent effects. Calendula-flower oil is a natural oil that is extracted from the flowers of marigolds and can be used as an anti-inflammatory agent, an anti-tumor agent, a remedy for healing wounds, and a remedy for other ailments. Menthol is an organic compound that is extracted from mint oils and can be used to trigger a cooling sensation on the skin. Peppermint oil is a natural oil that is extracted from a peppermint plant and can be used as a treatment for hemorrhoids. The combination of these ingredients provides an all natural healing salve that can be used to treat a variety of skin ailments such as psoriasis, eczema, cuts, burns, itching, chapped lips, blisters, dermatitis, and hemorrhoids.

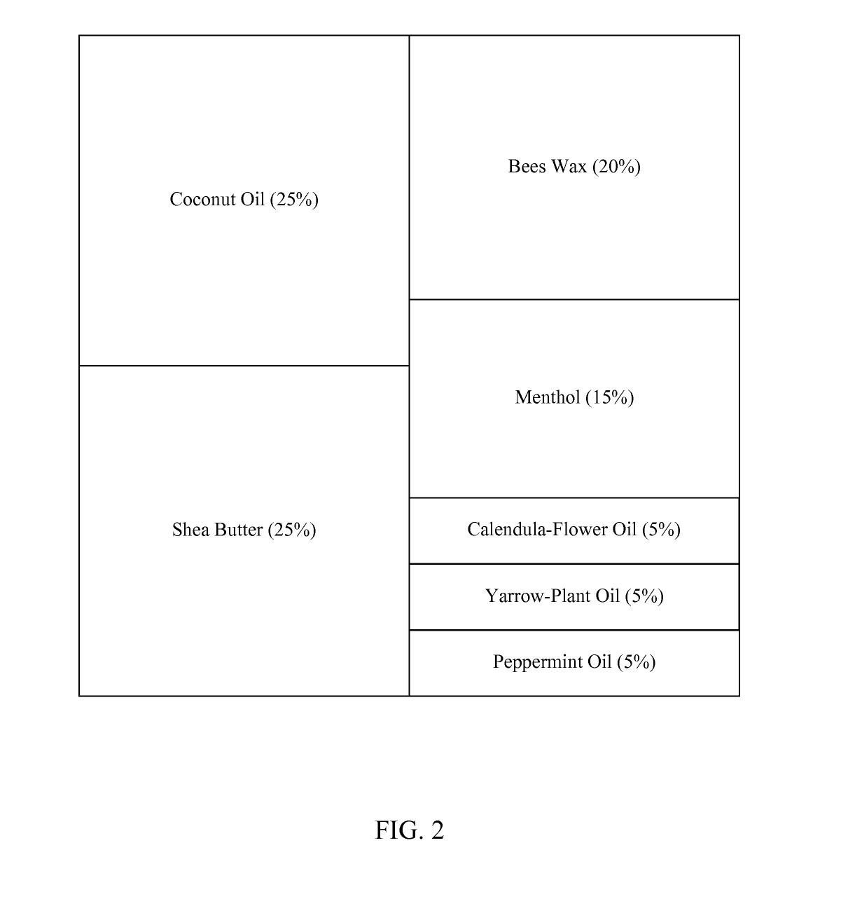

In the preferred embodiment of the present invention, each of the ingredients is combined at specific partitions, which is shown in FIG. 2. The quantity of bees wax is about 20%, by weight, of the caudal salve. The quantity of shea butter is about 25%, by weight, of the caudal salve. The quantity of the coconut oil is about 25%, by weight, of the caudal salve. Consequently, the quantity of bees wax, the quantity of shea butter, and the quantity of coconut oil are used as the base ingredients for the present invention. Moreover, the quantity of yarrow-plant oil is about 5%, by weight, of the caudal salve. The quantity of calendula-flower oil is about 5%, by weight, of the caudal salve. The quantity of menthol is about 15%, by weight, of the caudal salve. The quantity of peppermint oil is about 5%, by weight, of the caudal salve. The quantity of yarrow-plant oil, the quantity of calendula-flower oil, the quantity of menthol, and the quantity of peppermint oil are supplemental yet active ingredients of the present invention.

Bees wax works well within the present invention because it contains compounds called wax esters, which keeps skin hydrated. Bees wax can also be helpful as an ultraviolet light inhibitor and as a skin conditioner. Bees wax has special characteristics such as plasticity and compatibility that allow it to emulsify with other natural products and allow it to produce a pleasant scent. The quantity of bees wax used in the present invention can be either kind: white wax or yellow wax.

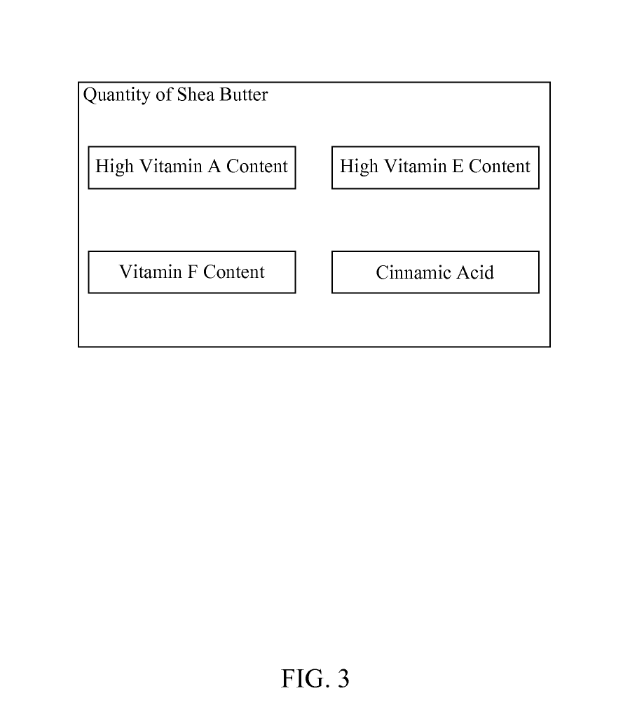

Shea butter works well within the present invention because it contains a high natural content of oil, which also keeps skin hydrated. Shea butter benefits those who are suffering from dry skin, dermatitis, sun burn, and a number of other skin ailments. However, shea butter is not known to trigger off any allergic reactions, which make it ideal for people with sensitive skin. In addition, the quantity of shea butter is known to comprise a plurality of topically beneficial constituents that include a high vitamin-E content, a high vitamin-A content, a vitamin-F content, and a cinnamic-acid content. These constituents are illustrated in FIG. 3. The high vitamin-E content and the high vitamin-A content allows the present invention to treat sun-damaged or wrinkled skin and keeps the skin healthy and supple. The vitamin-F content allows the present invention to rejuvenate rough, dry skin. Moreover, the quantity of shea butter used in the present invention is specifically a natural and organic substance.

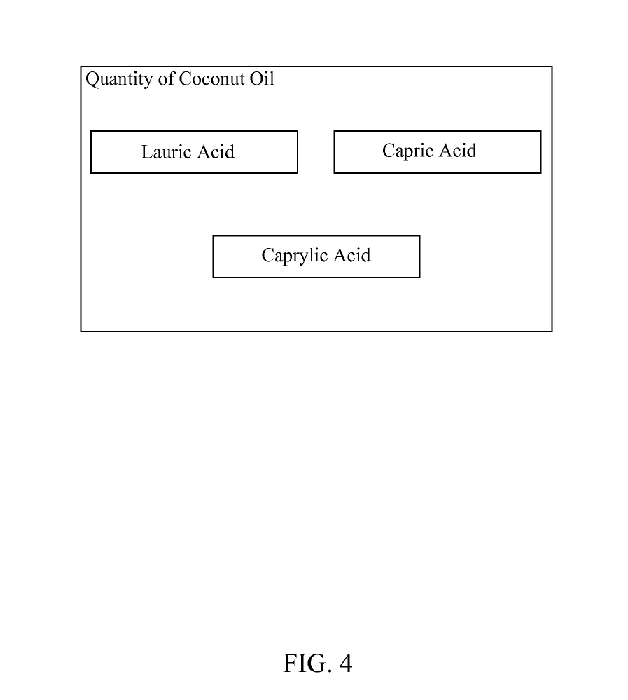

Coconut oil works well within the present invention because it contains high levels of saturated fat, which keeps skin hydrated. Coconut oil is helpful in nourishing damaged hair with essential proteins and is helpful in preventing protein-loss from both damaged and undamaged hair. Coconut oil is also helpful in preventing premature aging and degenerative diseases due to its antioxidant properties. Coconut oil can also be used to form a chemical layer over an infected body part, which allows the present invention to protect the infected body part from external dust, fungi, bacteria, and viruses. Coconut oil is most effective on speeding up the healing process for bruises. In addition, the quantity of coconut oil is known to comprise a plurality of topically beneficial constituents that include a lauric-acid content, a capric-acid content, and a caprylic-acid content. These constituents are illustrated in FIG. 4. The lauric-acid content, the capric-acid content, and the caprylic-acid content have antifungal, antibacterial, and antiviral properties, which could assist in disinfecting the area of the skin where the present invention is applied.

Yarrow-plant oil works well within the present invention because it has historically been used to treat external skin wounds. Yarrow-plant oil has antibiotic, anti-inflammatory, antiseptic, antispasmodic, astringent, and sedative properties. Thus, yarrow-plant oil allows the present invention to help heal bruises, burns, cuts, swelling, ulcers, and wounds on the skin or body surface. Yarrow-plant oil also allows the present invention to help deal with skin conditions such as eczema.

Calendula-flower oil works well within the present invention because it contains cosmetically beneficial compounds such as saponins, resins, and essential oils. Calendula-flower oil is a mild and soothing skin treatment for those with sensitive skin. For example, calendula oil can be used to treat diaper rash for babies. Calendula-flower oil also has antiseptic, anti-fungal, anti-inflammatory, and antibacterial properties, which allows the present invention to treat skin infections. In addition, calendula-flower oil can act as a moisturizer to repair dry or chapped skin and can be used to reduce the swelling of sprained muscles, bruises, spider veins, varicose veins, leg ulcers, and chilblains. Calendula-flower oil also increases collagen levels, which allows the present invention to prevent the formation of scar tissue.

Menthol works well within the present invention because it has the ability to chemically trigger the cold-sensitive receptors within the skin. Thus, when the present invention is applied to an irritated piece of skin, the menthol will provide a cooling sensation on the irritated piece of skin that should further comfort the user.

Similarly, peppermint oil also has the ability to chemically trigger cold-sensitive receptors within the skin and, thus, provides the same benefits to the present invention as menthol does. Peppermint oil also works well within the present invention because it includes the following minerals and nutrients: iron, magnesium, calcium, folate, manganese, copper, potassium, omega-3 fatty acids, vitamin A and vitamin C.

Although the invention has been explained in relation to its preferred embodiment, it is to be understood that many other possible modifications and variations can be made without departing from the spirit and scope of the invention as hereinafter claimed.

What is claimed is:

1. A caudal salve comprises:
a quantity of bees wax;
a quantity of shea butter;
a quantity of coconut oil;
a quantity of yarrow-plant oil;
a quantity of calendula-flower oil;
a quantity of menthol; and
a quantity of peppermint oil.

2. The caudal salve as claimed in claim 1 comprises:
said quantity of bees wax being about 20% wt of said caudal salve;
said quantity of shea butter being about 25% wt of said caudal salve;
said quantity of coconut oil being about 25% wt of said caudal salve;
said quantity of yarrow-plant oil being about 5% wt of said caudal salve;
said quantity of calendula-flower oil being about 5% wt of said caudal salve;
said quantity of menthol being about 15% wt of said caudal salve; and
said quantity of peppermint oil being about 5% wt of said caudal salve.

3. The caudal salve as claimed in claim 1 comprises:
said quantity of shea butter comprises a plurality of topically beneficial constituents; and
said plurality of topically beneficial constituents includes a high vitamin-A content, a high vitamin-E content, a vitamin-F content, and a cinnamic-acid content.

4. The caudal salve as claimed in claim 1 comprises:
said quantity of coconut oil comprises a plurality of topically beneficial constituents; and
said plurality of topically beneficial constituents includes a lauric-acid content, a capric-acid content, and a caprylic-acid content.

5. The caudal salve as claimed in claim 1, wherein the quantity of bees wax, the quantity of shea butter, and the quantity of coconut oil are base ingredients for the caudal salve.

6. The caudal salve as claimed in claim 1, wherein the quantity of yarrow-plant oil, the quantity of calendula-flower oil, the quantity of menthol, and the quantity of peppermint oil are supplemental yet active ingredients for the caudal salve.

7. The caudal salve as claimed in claim 1, wherein said quantity of shea butter being a natural and organic substance.

8. A caudal salve comprises:
a quantity of bees wax;
a quantity of shea butter;
a quantity of coconut oil;
a quantity of yarrow-plant oil;
a quantity of calendula-flower oil;
a quantity of menthol;
a quantity of peppermint oil;
said quantity of bees wax being about 20% wt of said caudal salve;
said quantity of shea butter being about 25% wt of said caudal salve;
said quantity of coconut oil being about 25% wt of said caudal salve;
said quantity of yarrow-plant oil being about 5% wt of said caudal salve;
said quantity of calendula-flower oil being about 5% wt of said caudal salve;
said quantity of menthol being about 15% wt of said caudal salve; and
said quantity of peppermint oil being about 5% wt of said caudal salve.

9. The caudal salve as claimed in claim 8 comprises:
said quantity of shea butter comprises a plurality of topically beneficial constituents; and
said plurality of topically beneficial constituents includes a high vitamin-A content, a high vitamin-E content, a vitamin-F content, and a cinnamic-acid content.

10. The caudal salve as claimed in claim 8 comprises:
said quantity of coconut oil comprises a plurality of topically beneficial constituents; and
said plurality of topically beneficial constituents includes a lauric-acid content, a capric-acid content, and a caprylic-acid content.

11. The caudal salve as claimed in claim 8, wherein the quantity of bees wax, the quantity of shea butter, and the quantity of coconut oil are base ingredients for the caudal salve.

12. The caudal salve as claimed in claim 8, wherein the quantity of yarrow-plant oil, the quantity of calendula-flower oil, the quantity of menthol, and the quantity of peppermint oil are supplemental yet active ingredients for the caudal salve.

13. The caudal salve as claimed in claim 8, wherein said quantity of shea butter being a natural and organic substance.

14. A caudal salve comprises:
a quantity of bees wax;
a quantity of shea butter;
a quantity of coconut oil;
a quantity of yarrow-plant oil;
a quantity of calendula-flower oil;
a quantity of menthol;
a quantity of peppermint oil;
said quantity of bees wax, said quantity of shea butter, and said quantity of coconut oil being base ingredients for said caudal salve; and
said quantity of yarrow-plant oil, said quantity of calendula-flower oil, said quantity of menthol, and said quantity of peppermint oil are supplemental yet active ingredients for said caudal salve.

15. The caudal salve as claimed in claim 14 comprises:
said quantity of bees wax being about 20% wt of said caudal salve;
said quantity of shea butter being about 25% wt of said caudal salve;
said quantity of coconut oil being about 25% wt of said caudal salve;
said quantity of yarrow-plant oil being about 5% wt of said caudal salve;
said quantity of calendula-flower oil being about 5% wt of said caudal salve;
said quantity of menthol being about 15% wt of said caudal salve; and
said quantity of peppermint oil being about 5% wt of said caudal salve.

16. The caudal salve as claimed in claim 14 comprises:
said quantity of shea butter comprises a plurality of topically beneficial constituents; and
said plurality of topically beneficial constituents includes a high vitamin-A content, a high vitamin-E content, a vitamin-F content, and a cinnamic-acid content.

17. The caudal salve as claimed in claim 14 comprises:
said quantity of coconut oil comprises a plurality of topically beneficial constituents; and
said plurality of topically beneficial constituents includes a lauric-acid content, a capric-acid content, and a caprylic-acid content.

18. The caudal salve as claimed in claim 14, wherein said quantity of shea butter being a natural and organic substance.
